(19) United States
(12) Patent Application Publication
(10) Pub. No.: US 2023/0256219 A1
(43) Pub. Date: Aug. 17, 2023

(54) BIODEGRADABLE MICRONEEDLE PATCH FOR TRANSDERMAL GENE DELIVERY

(71) Applicant: THE REGENTS OF THE UNIVERSITY OF CALIFORNIA, Oakland, CA (US)

(72) Inventors: Alireza Khademhosseini, Los Angeles, CA (US); Wujin Sun, Los Angeles, CA (US)

(73) Assignee: THE REGENTS OF THE UNIVERSITY OF CALIFORNIA, Oakland, CA (US)

(21) Appl. No.: 18/007,039

(22) PCT Filed: Jul. 28, 2021
(86) PCT No.: PCT/US2021/043577

§ 371 Date: Jan. 26, 2023

Related U.S. Application Data
(60) Provisional application No. 63/062,304, filed on Aug. 6, 2020.

(51) Int. Cl. A61M 37/00; A61K 9/00; A61K 47/69

(52) U.S. Cl. CPC A61M 37/0015; A61K 9/0021; A61K 47/6949; A61M 2037/0023; A61M 2037/0061

(57) ABSTRACT

A biodegradable microneedle (MN) patch is disclosed for transdermal gene delivery. The MN patch, in one embodiment, is a gelatin methacryloyl (GelMA)-based MN platform for the local and controlled transdermal delivery of plasmid DNA (pDNA) (or other nucleic acid with high transfection efficiency both in vitro and in vivo. Intracellular delivery of the nucleic acid cargo is enabled by poly(β-amino ester) (PBAE). After being embedded in the GelMA MNs, sustained release of DNA-encapsulated PBAE nanoparticles (NPs) is achieved and the release profiles can be controlled by adjusting the degree of crosslinking of the GelMA. These results highlight the advantages and potential of using PBAE/DNA NPs embedded GelMA MN patches (MN/PBAE/DNA) for successful transdermal delivery of pDNA for tissue regeneration, cancer therapy, and other applications. The patch may also be used on other tissue types.

### RELATED APPLICATION

This Application claims priority to U.S. Provisional Pat. Application No. 63/062,304 filed on Aug. 6, 2020, which is hereby incorporated by reference. Priority is claimed pursuant to 35 U.S.C. § 119 and any other applicable statute.

### TECHNICAL FIELD

The technical field generally relates to microneedle (MN) patches for the transdermal delivery of nucleic acid cargoes. More particularly, the technical field generally relates to MN patches for the local and controlled transdermal delivery of nucleic acid with high transfection efficiency both in vitro and in vivo. Intracellular delivery of the nucleic acid cargo is enabled by the addition of gene delivery nanoformulations. As specific embodiment uses poly(β-amino ester) (PBAE) and plasmid DNA (pDNA).

### STATEMENT REGARDING FEDERALLY SPONSORED RESEARCH AND DEVELOPMENT

This invention was made with government support under Grant Number GM126831, awarded by the National Institutes of Health. The government has certain rights in the invention.

### BACKGROUND

Gene therapies can treat diseases by delivering exogenous nucleic acid into cells to express functional and therapeutic proteins. Gene delivery has been applied to cancer therapy and tissue engineering in which the delivered genes can change properties of tumor cells, immune cells, and stem cells. With the FDA approval of anti-CD19 chimeric antigen receptor (CAR) T-cell therapy and LUXTURNA®, an adeno-associated virus vector-based gene therapy for biallelic RPE65 mutation-associated retinal dystrophy, many strategies for high efficiency gene delivery are being investigated. However, major challenges including the development of suitable delivery carriers and the identification of appropriate delivery routes still exist for gene therapy.

Gene delivery vectors primarily fall into two categories: viral and non-viral. Biosafety concerns of viral vectors, delivery methods based on the use of viruses, pose challenges in further applying them in human. Non-viral vectors, methods that do not rely on viruses, are one promising approach to mitigate adverse immune responses and safety concerns. Systemic delivery of genes through non-viral carriers is another approach that is complicated by off-target side effects or nucleic acid stability in biological fluids. Furthermore, the immune system can recognize and destroy vectors containing genetic information. The local delivery of genetic therapeutics can overcome the limitations of systemic delivery and achieve targeted transfection in a precise manner. The epidermis is enriched with vasculature, lymph ducts, and a diverse population of immune cells. These properties make the skin an ideal target for the delivery of nucleic acids as treatments for genetic defects, cutaneous cancers, hyperproliferative diseases, wounds, and infections. However, the epidermis is a barrier that limits the availability of therapeutics.

Microneedle (MN) patches have been widely studied for transdermal drug delivery because they are capable of penetrating the stratum corneum to enhance local drug delivery with minimal pain and improved patient compliance. MNs, such as metal MNs, coated MNs, and dissolving/biodegradable MNs, are promising tools for gene delivery as they can permeabilize restrictive tissue barriers. Compared with coated MNs, dissolvable and biodegradable MNs can achieve sustained and controlled release, thus avoiding the side effects caused by burst release of genetic therapeutics.

### SUMMARY

In one embodiment, a gelatin methacryloyl (GelMA) microneedle (MN)-based platform for local and controlled transdermal delivery of plasmid DNA (pDNA) is disclosed with high transfection efficiency both in vitro and in vivo. The platform is the form a GelMa-based MN patch for the delivery of nucleic acid. GelMA is derived from natural gelatin with photocrosslinkable, biodegradable, and biocompatible features. Here, a biodegradable patch is disclosed that uses GelMA-based MNs that can be loaded with genetic material for transdermal transfection purposes. Intracellular delivery of the nucleic acid cargo is enabled by poly(β-amino ester) (PBAE) nanoparticles (NPs). After being embedded in the GelMA MNs, sustained release of DNA-encapsulated PBAE nanoparticles (NPs) is achieved and the release profiles can be controlled by adjusting the degree of crosslinking of the GelMA that forms the MNs. These results highlight the advantages and potential of using PBAE/DNA NPs embedded GelMA MN patches (MN/PBAE/DNA) for successful transdermal delivery of pDNA for applications such as, for example, tissue regeneration and cancer therapy.

To enhance the genetic material uptake and function without compromising biocompatibility, a synthetic cationic polymer, poly(β-amino ester) (PBAE) was selected, that is able to complex with negatively charged plasmid DNA (pDNA). The biodegradability of PBAE facilitates efficient gene delivery while avoiding the induction of an inflammatory response or cytotoxicity associated with synthetic polymers that cannot be degraded.

In one embodiment, a patch for nucleic acid delivery across a biological barrier of living tissue includes a base or substrate having a plurality of microneedles extending away from the surface of the base, wherein the base and the plurality of microneedles are formed from crosslinked gelatin methacryloyl (GelMA) and the plurality of microneedles contain that contains poly(β-amino ester) (PBAE)/nucleic acid nanoparticles (NPs) therein.

In one embodiment, a patch for nucleic acid delivery into living tissue includes a base or substrate having a plurality of microneedles extending away from the surface of the base, wherein the base and the plurality of microneedles are formed from crosslinked gelatin methacryloyl (GelMA) and the plurality of microneedles contain therein gene delivery nanoformulation(s) and nucleic acid.

In another embodiment, a patch for nucleic acid delivery into living tissue includes a base or substrate having a plurality of microneedles extending away from the surface of the base, wherein the base and the plurality of microneedles are formed from crosslinked gelatin methacryloyl (GelMA) and the plurality of microneedles contain that contains nanoparticles (NPs) therein, the NPs formed a polyplex between a nucleic acid carrier and a nucleic acid.

In another embodiment, a method of using the patch includes placing the patch on live tissue of mammal such that the plurality of microneedles penetrates into the tissue. In one embodiment the tissue is skin tissue. In other embodiments, the tissue includes mucosal tissue, heart tissue, blood vessels, ocular tissue, gastrointestinal tissue, buccal tissue, muscle tissue, and vaginal tissue.

In another embodiment, a method of manufacturing a patch for gene delivery across a biological barrier includes: providing a mold containing a plurality of needle shaped cavities therein; applying a solution of gelatin methacryloyl (GelMA), poly(β-amino ester) (PBAE)/nucleic nanoparticles (NPs), and a photoinitiator on or surrounding the mold; subjecting the mold to centrifugation or vibration; irradiating the mold containing the solution with light to crosslink the GelMA; and removing the patch from the mold.

### BRIEF DESCRIPTION OF THE DRAWINGS

FIG. 1A illustrates a plan view of a patch for transdermal gene delivery according to one embodiment. The patch includes a base or substrate and a plurality of microneedles that extend from a surface thereof.

FIG. 1B illustrates a cross-sectional view of a patch illustrating the base or substrate and the plurality of microneedles that extend from a surface thereof. Nucleic acid (e.g., DNA) is illustrated being disposed within the microneedles and the base or substrate.

FIG. 1C illustrates a patch for transdermal gene delivery being applied to skin tissue of a mammal (e.g., human).

FIG. 1D illustrates a plan view of an alternative embodiment of a patch for transdermal gene delivery.

FIGS. 2A-2C show a schematic for the components and application of MN/PBAE/DNA. FIG. 2A shows an 11 × 11 GelMA MN array embedded with PBAE/DNA NPs can be applied as transdermal gene delivery device. FIG. 2B illustrates how a synthesized PBAE can complex with pDNA to achieve NP self-assembly by charge attraction. Freshly prepared PBAE/DNA NPs can be embedded in GelMA solutions to fabricate gene delivery MN patches with a mold-based casting method. FIG. 2C is a schematic for the transdermal gene transfection with MN/PBAE/DNA. After applying the MN/PBAE/DNA to the skin, the PBAE/DNA NPs can be released with the degradation of the GelMA, which can deliver exogenous nucleic acids into cells.

FIGS. 3A-3E illustrate the characterization of PBAE/DNA NPs with different ratios of PBAE/DNA (w/w). FIG. 3A illustrates the size and zeta-potential of PBAE/DNA NPs with predetermined ratios of PBAE/DNA (w/w). FIG. 3B shows fluorescence microscopy images and flow-cytometry analysis of treated cells using free DNA, Lipofectamine, and PBAE/pEGFP NPs with predetermined ratios of PBAE/DNA (w/w). Cell nucleus and dead cells were labelled with DAPI and EthD-1, respectively. Scale bar: 100 µm. (Fluorescence and flow cytometry results of 40/1, 60/1, and 100/1 group are shown in FIG. 10.) FIG. 3C shows a graph of the transfection efficiencies of each group were analyzed by flow cytometry. FIG. 3D illustrates a graph of the viabilities of treated cells in each group at predetermined time points analyzed by CCK-8 assay. FIG. 3E shows the diameter distribution and TEM image of PBAE/pEGFP NPs with the ratio of PBAE/DNA (w/w) at 80/1. Scale bar: 200 nm. Data are shown as mean ± SD (n = 3). (*, p < 0.05; **, p < 0.01; ***, p < 0.001).

FIGS. 4A-4I illustrate the characterization of the MN/PBAE/DNA. FIG. 4A is an image showing the gross appearance of MN/PBAE/DNA patch with a 10 s crosslinking time. FIG. 4B and FIG. 4C show SEM images of MN/PBAE/DNA with a 10 s crosslinking time. Scale Bar: 500 µm and 100 µm, respectively. FIG. 4D and FIG. 4E are high magnification SEM images to show the surface detail of MN/PBAE/DNA and blank MN, respectively. Arrows indicate PBAE/DNA NPs embedded in MN/PBAE/DNA which can be observed. Scale bar: 3 µm. FIG. 4F shows ex-vivo transdermal test using MN/PBAE/DNA with 10 s crosslinking time. Scale bar: 500 µm. FIG. 4G us a graph showing the mechanical property of the MN/PBAE/DNA patch with predetermined crosslinking time. Blank MN patches were used as a control. FIG. 4H and FIG. 4I illustrate PBAE/DNA NP release profiles from MN/PBAE/DNA with (FIG. 4H) or without collagenase (FIG. 4I), respectively. Data are shown as mean ± SD (n = 3).

FIGS. 5A-5C illustrate gene transfection in 2D cultured fibroblasts with MN/PBAE/DNA for 72 h. FIG. 5A is a schematic for the experimental design. FIG. 5B shows fluorescence microscopy images and flow cytometry analysis of cells treated with MN/DNA and MN/PBAE/DNA with different crosslinking times. Cell nuclei and dead cells were labelled with DAPI and EthD-1, respectively. Scale bar: 100 µm. FIG. 5C shows transfection rates of 2D cell transfection with MN/DNA and MN/PBAE/DNA with different crosslinking time based on the results of flow cytometry. Data are shown as mean ± SD (n=3). (*, p < 0.05; **, p < 0.01; ***, p < 0.001)

FIGS. 6A and 6B show gene transfection in 3D cultured fibroblasts with MN/PBAE/DNA with 10 s crosslinking. FIG. 6A is a schematic for the experimental design. MN/PBAE/DNA were applied to a hydrogel matrix loaded with cells to release PBAE/DNA NPs for transfection; FIG. 6B shows confocal images of the treated cells, cell nuclei and dead cells were labelled with DAPI and EthD-1, respectively.

FIG. 7 illustrates in vivo transdermal gene transfection using MN/PBAE/DNA. In HE stained and immunofluorescence-stained images, it can be observed that all MN patches penetrated the epidermal layer and the MN/PBAE/DNA achieved the greatest cell transfection in the dermis compared to Blank MNs and MN/DNA without significant inflammation. Transfection rate of cells in boxed region of interest was ~31%. Scale bar: 1 mm, 100 µm, and 100 µm, respectively.

FIGS. 8A-8C illustrate ¹H-NMR spectra of reactants and products in the synthesis of PBAE. FIG. 8A shows ¹H-NMR spectra of 5-amino-1-pentanol. FIG. 8B shows ¹H-NMR spectra of 1,4-butanediol diacrylate. FIG. 8C shows ¹H-NMR spectra of synthesized poly(5-amino-1-pentanol-co-1,4-butanediol diacrylate). The disappearance of peak b, c, and d and the presence of peak a indicate the successful synthesis of PBAE.

FIG. 9 illustrates agarose gel electrophoresis of PBAE/DNA NPs with predetermined ratios of PBAE/DNA (w/w).

FIG. 10 illustrates in vitro gene transfection using PBAE/pEGFP NPs with predetermined ratios of PBAE/DNA (w/w). Transfection rate and cytotoxicity of each group were analyzed by flow cytometry (Fluorescence and flow cytometry results of free DNA, 80/1, and Lipofectamine group are shown in FIG. 3B).

FIG. 11 illustrates relative fluorescent intensity of DNA solutions quantified with PicoGreen®. The binding of DNA with PBAE in the formation of PBAE/DNA NPs affected the binding and coloration of PicoGreen®. Heparin can disrupt the polyplexes and completely free the DNA when the mass ratio of heparin/PBAE was over 0.5:1. Data are shown as mean ± SD (n = 3). (*, p < 0.05; **, p < 0.01; ***, p < 0.001).

FIG. 12 illustrates individual channel and merged images of transfected cells of each group in 2D cell transfection with MN/PBAE/DNA. Dead cells and cell nuclei were labeled with EthD-1 and DAPI, respectively. Scale bar: 100 µm.

### DETAILED DESCRIPTION OF ILLUSTRATED EMBODIMENTS

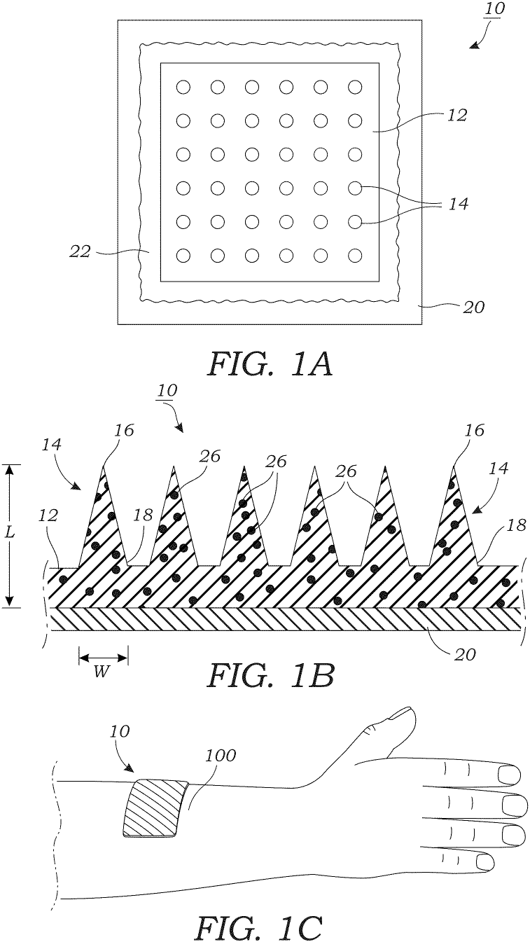

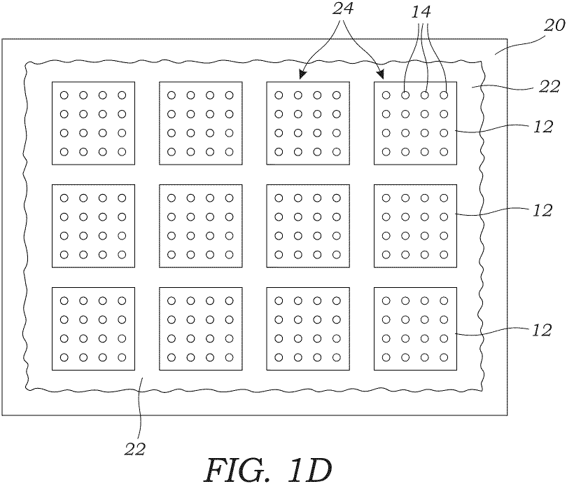

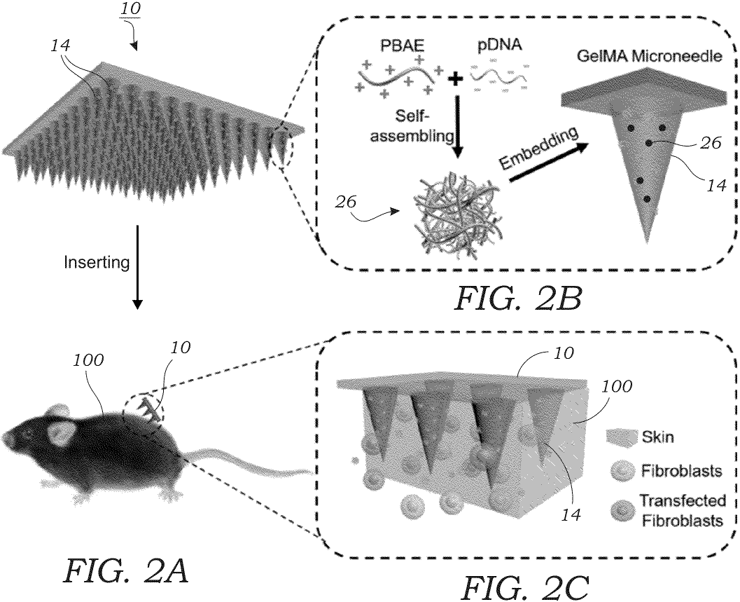

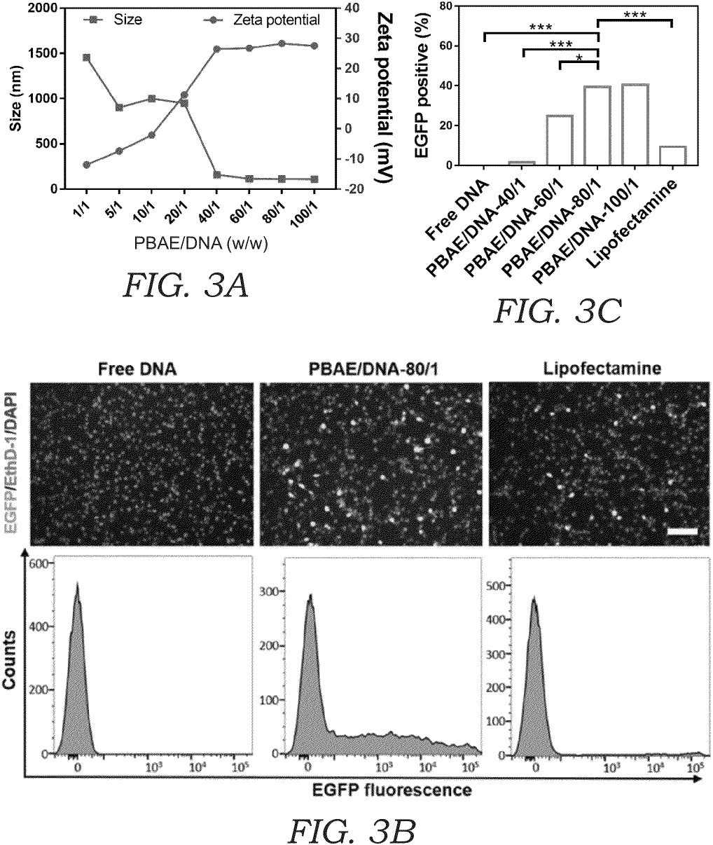

FIG. 1A illustrates plan view of a patch 10 for transdermal nucleic acid (e.g., gene or gene fragment) delivery according to one embodiment. Nucleic acids include DNA and RNA. This may include plasmid DNA (pDNA), messenger RNA (mRNA), small interfering RNA (siRNA), micro RNA (miRNA), and the like. The patch 10 includes a base or substrate 12 that includes a plurality of microneedles 14 that extend or project from the substrate 12. The patch 10 may in some embodiments be partly or entirely biodegradable. The term biodegradable in the context of a biodegradable patch 10 refers to the base or substrate 12 and/or the microneedles 14 being formed from a material that is biodegradable. Other components such as the optional backing material 20 discussed below may not be biodegradable yet the patch 10 may still be referred to as being “biodegradable.” The plurality of microneedles 14 generally extend or project in a perpendicular direction from a surface of the base or substrate 12. The plurality of microneedles 14 may be arranged in a regular repeating array as illustrated in FIG. 1A or, alternatively, they may be arranged in a random pattern. In one embodiment, the plurality of microneedles 14 that are formed on the base or substrate 12 may have substantially similar shapes and sizes. However, in other embodiments, the plurality of microneedles 14 may have different shapes and/or sizes. For example, the perimeter region of the array or field of microneedles 14 that extend from the base or substrate 12 may be longer or have different shapes than those in the central region of the patch 10 to better secure the patch 10 to site of application.

In one particular embodiment, the microneedles 14, as their name implies, have a needle-like shape. For example, the microneedles 14 may include a sharpened tip 16 (seen in FIG. 1B) that aid in penetrating the epidermal layer of the skin tissue 100 (seen in FIG. 1C). The length (L) of the microneedles 14 may vary although typically the microneedles 14 extend less than about 1.5 mm from the base or substrate 12 (FIG. 1B). A typical length of the microneedles 14 is around 300-700 µm, although the dimensions may extend outside this range (e.g., around 10 µm to around 1,500 µm). The base 18 of the microneedle 14 is wider than the tip 16. Typically, the base 18 of the microneedle 14 may have a diameter or width (W) that is less than about 500 µm (e.g., 300 µm base and a height of around 600-700 µm) (FIG. 1B). The distance between adjacent microneedles 14 (i.e., pitch) may be several hundred micrometers (e.g., about 300 µm). The particular dimensions and shape(s) of the microneedles 14 are controlled by the particular construction of the mold that is used to form the patch 10.

Still referring to FIGS. 1A and 1B, the base or substrate 12 which holds the microneedles 14 may be bonded or otherwise adhered to an optional backing material 20 (e.g., through the use of an adhesive, chemical linking, or the like). The backing material 20 may be made from a woven fabric, a plastic material such as polyvinylchloride, polyethylene, or polyurethane, or latex. The backing material 20 may be flexible so that the patch 10, when applied, can conformally cover the tissue 100 (seen in FIG. 1C). Optionally, the backing material 20 may include an adhesive material 22 that covers all or a portion of the tissue-facing surface of the backing material 20. For example, adhesive may be formed on the backing material 20 around the periphery of the base or substrate 12 or the backing material 20 so that the base or substrate 12 may be secured in place to the surface of the tissue 100. The adhesive material 22 aids in securing the patch 10 to the tissue 100. The adhesive material 22 may include resins (e.g., vinyl resins), acrylates such as methacrylates epoxy diacrylates. As an alternative to the backing material 20, the patch 10 may be secured to the tissue 100 using a bandage or wrap. The patch 10 may also just be mechanically secured to the tissue 100 using the microneedles 14.

The base or substrate 12 and the microneedles 14 may be somewhat rigid in the dry state. Because of this, in one alternative embodiment which is illustrated in FIG. 1D, multiple sub-patches 24 may be integrated into the backing material 20 to make the final patch 10. This may be useful for large coverage areas or curved surfaces that may pose a risk of breakage to the base or substrate 12. The various sub-patches 24, while generally rigid, are still able to conform to the surface of the tissue 100 (e.g., FIG. 1C) due the flexible backing material 20 which enables bending of the overall patch 10. Because individual sub-patches 24 are smaller in size these do not experience significant bending stresses which would otherwise cause a larger, rigid structure to break in response to bending and/or manipulation. Bending or flexing can occur within the backing material 20 between the locations of where the sub-patches 24 are located (e.g., between the rows and columns of sub-patches 24).

In one embodiment, with reference to FIG. 1B, the base or substrate 12 and the plurality of microneedles 14 are formed from crosslinked GelMA material that contains nucleic acid in combination with a gene delivery nanoformulation. In one specific embodiment, the crosslinked GelMA contains poly(β-amino ester) (PBAE)/nucleic acid nanoparticles (NPs) 26 therein (e.g., DNA as the nucleic acid). The PBAE/nucleic acid NPs 26 in this embodiment function as the gene delivery nanoformulation. The NPs 26 are in the form of a polyplex which is a nanometer-sized complex formed through electrostatic interaction of cationic polymers and nucleic acids (e.g., one specific example of a gene delivery nanoformulation). The cationic polymer PBAE is able to complex with the negatively charged DNA (e.g., plasmid DNA or pDNA) for form the NPs 26. The PBAE/DNA NPs 26 may be dispersed throughout the entirety of the patch 10 including the base or substrate 12 and the plurality of microneedles 14 although in other embodiments the PBAE/DNA NPs 26 may be located only in the microneedles 14. The biodegradable GelMA matrix used for the patch 10 also serves as a protective scaffold for the PBAE/nucleic acid NPs 26 in which the release profile of the NPs 26 can be controlled by the degree of crosslinking in the hydrogel. While one embodiment of the NPs 26 uses a PBAE/DNA-based polyplex, other polyplex NPs 26 are also contemplated. These include by way of example, poly(ethyleneimine) and poly(lysine) based NPs 26. Other gene delivery nanoformulations may also be used besides polyplexes. These include, by way of example, lipoplex, liposomes, peptides, dendrimers, and the like. These nanoformulations would also be contained in the microneedles 14 and/or base or substrate 12 of the patch.

As explained herein, the base or substrate 12 and the microneedles 14 are preferably made from crosslinked GelMA. GelMA is a derivative of gelatin with modified methacrylamide or methacrylate groups. GelMA may be crosslinked by ultra-violet (UV) or visible light in the presence of a photoinitiator. It is a highly biocompatible material that is commonly used to support cell growth in tissue engineering. The existence of peptide moieties like arginine-glycine-aspartic acid (RGD) for cell attachment as well as for protease degradation makes GelMA a close mimic of the natural extracellular matrix (ECM). In addition, GelMA is a versatile material that can be easily functionalized with various bio-functionalities, such as by encapsulating different molecules including therapeutic agents, growth factors, and cytokines.

The microneedles 14 may have a number of different shapes and configurations including, for example, a pyramid, cone, cylindrical, tapered tip, canonical, square base, pentagonal-base canonical tip, and the like. The plurality of microneedles 14 swell upon breaching or penetrating the biological barrier and absorbing fluid from the surrounding tissue 100. The microneedles 14 may swell from about 100% to about 300% (wt. basis). The microneedles 14 swell and, in one embodiment, form a flexible hydrogel. The microneedles 14 provide a path for the PBAE/DNA NPs 26 to pass through the biological barrier (i.e., tissue 100). In some embodiments, the microneedles 14 are also biodegradable and dissolve over time.

The patch 10 is manufactured or fabricated by providing a mold (e.g., micro-mold) containing a plurality of needle shaped cavities therein. For example, the mold may be formed from a polymer such as polydimethylsiloxane (PDMS). Commercially available microneedle molds such as those made by Blueacre Technology Ltd. (Dundalk, Co Louth, Ireland) may be used. The GelMa is formed using established protocols such as those disclosed in Yue, K., et al., Structural analysis of photocrosslinkable methacryloyl-modified protein derivatives. Biomaterials, 2017. 139: p. 163-171, and Yue et al., Synthesis, properties, and biomedical applications of gelatin methacryloyl (GelMA) hydrogels, Biomaterials, 2015; p. 254-271, which are incorporated herein by reference. Details regarding the formation of GelMa is described in detail herein.

To use the patch 10, the patch 10 is applied to tissue 100 such as skin tissue. The microneedles 14 penetrate the skin tissue 100 and deliver the PBAE NPs 26 and nucleic acid (e.g., DNA/gene(s)) in a transdermal fashion. The biodegradability of PBAE facilitates efficient gene delivery while avoiding the induction of an inflammatory response or cytotoxicity associated with synthetic polymers. Of course, in other embodiments, a different specific gene delivery nanoformulation is loaded in the microneedles 14 and delivered to the tissue 100. This may include other types of NPs 26 such as, for example, poly(ethyleneimine) and poly(lysine) based NPs 26. Other gene delivery nanoformulations may also be used besides polyplexes. These include, by way of example, lipoplex, liposomes, peptides, dendrimers, and the like. These nanoformulations are contained in the microneedles 14 and/or base or substrate 12 of the patch 10 and released into the tissue 100. While skin tissue 100 is illustrated it should be appreciated that the patch 10 may be applied to other organs and/or tissue types. For example, exemplary tissue 100 includes mucosal tissue, heart tissue, blood vessels, ocular tissue, gastrointestinal tissue, buccal tissue, muscle tissue, and vaginal tissue as examples.

### Experimental

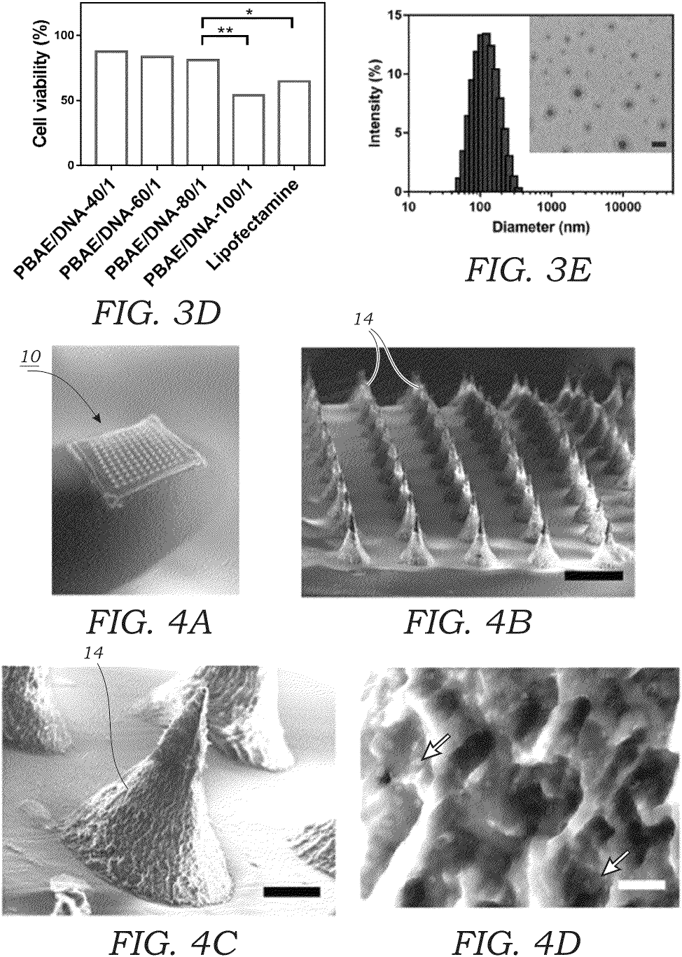

A transdermal MN patch 10 is disclosed to deliver PBAE/DNA nanoparticles (NPs) 26 embedded within crosslinkable GelMA matrix. GelMA MNs 14 can penetrate the epidermis to reach the targeted cells (FIGS. 2A-2C). In addition, the biodegradable GelMA matrix also serves as a protective scaffold for the PBAE/DNA NPs 26 in which the release profile of the NPs 26 can be controlled by the degree of crosslinking of the hydrogel. With plasmid DNA encoding EGFP (pEGFP) as the model cargo, the release profile of PBAE/DNA NPs 26 and local gene transfection efficiency were investigated with PBAE/DNA embedded GelMA MN patches 10(MN/PBAE/DNA) both in vitro and in vivo.

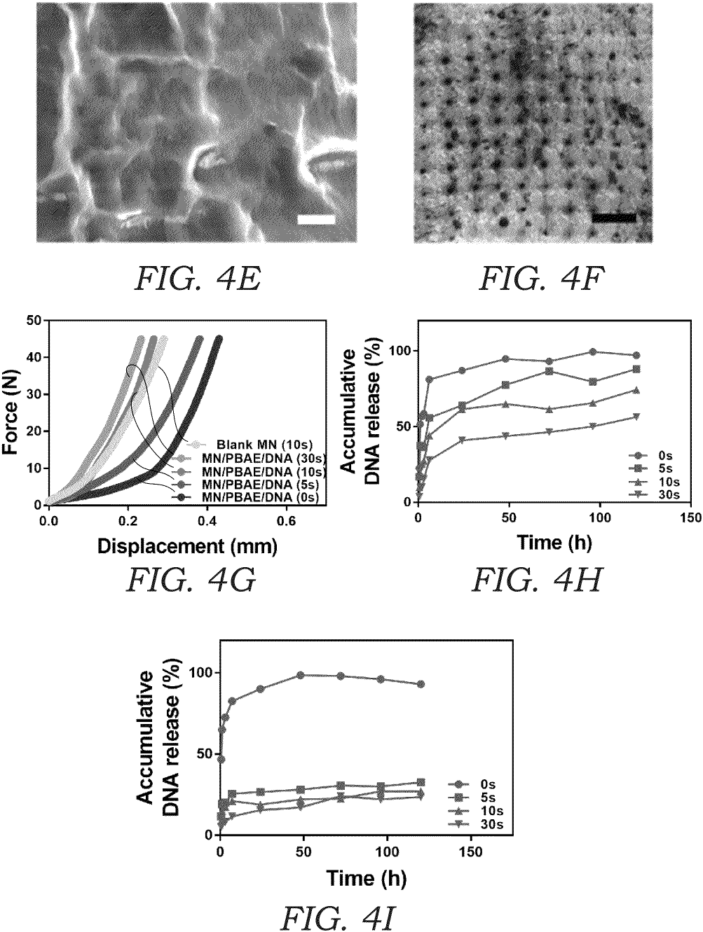

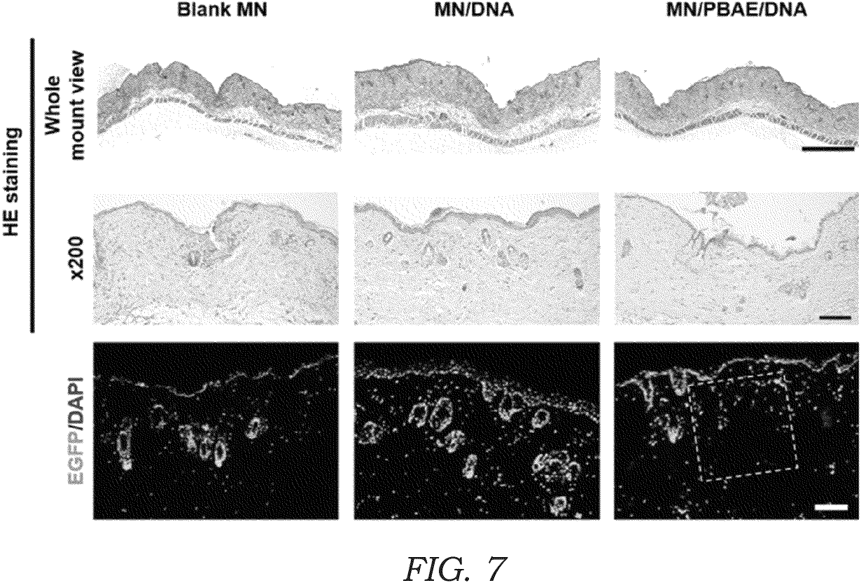

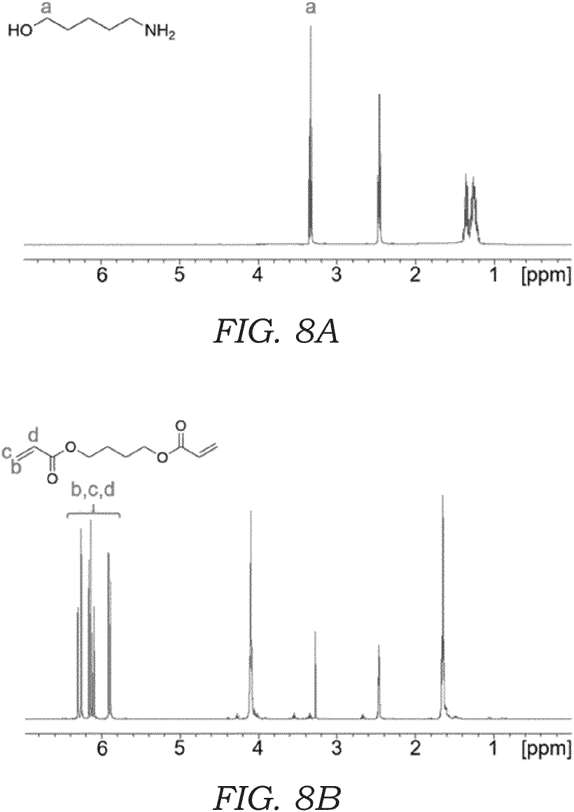

For the purpose of gene transfection, poly(5-amino-1-pentanol-co-1,4-butanediol diacrylate) was first synthesized as a gene delivery vector according to an established protocol as disclosed in D. G. Anderson et al., Mol Ther, 2005, 11, 426-434 and Y. Liu et al., Mol Pharm, 2018, 15, 4558-4567, which are incorporated by reference herein. The proton nuclear magnetic resonance (¹H-NMR) spectra of the reactants and products are shown in FIGS. 8A-8C. To optimize the NPs 26, PBAE and pDNA were incubated at different weight ratios (1:1, 5:1, 10:1, 20:1, 40:1, 60:1, 80:1, and 100:1) to identify the ideal ratio to form complexes. The PBAE/DNA complexes with the greatest delivery efficiency had diameters less than 250 nm and positive zeta-potentials in 10 mM HEPES buffer. As shown in FIG. 3A, the average size of the NPs 26 was less than 200 nm and the zeta-potential was observed to be ~ 29 mV when the PBAE/DNA ratio was greater than 40/1 (w/w). To further characterize the interactions between PBAE and encapsulated pDNA, a DNA electrophoresis assay was performed to determine the threshold of DNA charge neutralization by PBAE. By performing agarose gel electrophoresis, macromolecules are separated based on their charge and molecular weight. Using this principle, charged DNA contained within an agarose gel in the presence of an electric field and variable polycation concentration can be used to determine the critical point at which the negative charge of the DNA has been neutralized. As shown in FIG. 9, the retardation of DNA migration was observed from PBAE/DNA ratio as low as 1/1 (w/w) and DNA migration was completely inhibited at PBAE/DNA ratios above 20/1.

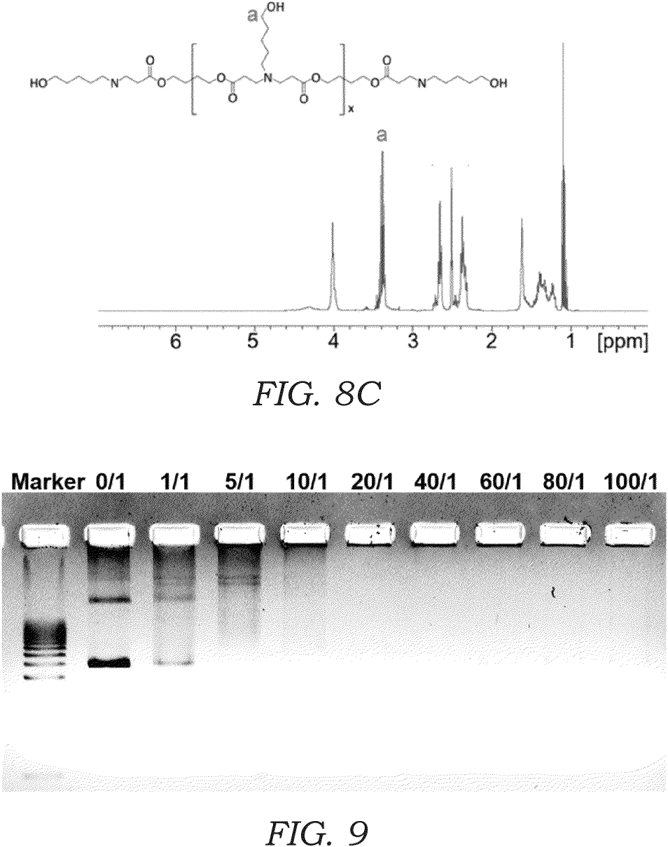

To further optimize the PBAE/DNA ratio for gene transfection, NPs 26 with PBAE/DNA ratios of 40:1, 60:1, 80:1, and 100:1 (w/w) were selected based on previous characterization. The gene transfection efficiency was evaluated using NIH 3T3 cells in vitro, and commercialized non-viral vector, Lipofectamine 3000, was used as control following instructions without further optimization. After treating with PBAE/pEGFP NPs at pre-determined PBAE/DNA ratios or Lipofectamine 3000 for 24 h, EGFP expression were quantified by flow cytometry. As shown in FIGS. 3B, 3C, and FIG. 10, PBAE/DNA at a weight ratio of 80/1 leads to significantly higher transfection efficiency (~ 40%) compared to the control groups. To evaluate cytotoxicity, the cell viability of each sample was tested using CCK-8 assay. As shown in FIG. 3D, the relative viability of cells treated with PBAE/DNA NPs 26 at a weight ratio of 80/1 was above 80% without significant difference from the 40/1 and 60/1 groups. The size distribution and morphology of NPs 26 with an 80/1 PBAE/DNA weight ratio were further characterized by dynamic light scattering (DLS) and transmission electron microscope (TEM) (FIG. 3E). In summary, PBAE/DNA NPs 26 with an 80/1 PBAE/DNA weight ratio outperformed other ratios due to its high transfection efficiency and biocompatibility and was selected for further integration. Thus, in one embodiment, the nanoparticles 26 have a ratio of PBAE to nucleic acid of about 80/1 on a weight basis. It should be appreciated that other ratios of PBAE to nucleic acid may also be used. This includes, for example, about 60/1 PBAE:nucleic acid, 65/1 PBAE:nucleic acid, 70/1 PBAE:nucleic acid, about 75/1 PBAE:nucleic acid, etc. For example, between about 60/1 and 80/1 may also provide high enough transfection efficiency and biocompatibility.

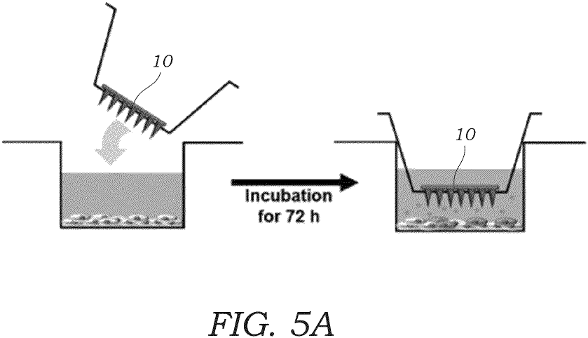

In the preparation of PBAE/DNA NPs 26, it was found the NPs 26 aggregated when DNA concentrations were above 80 µg/ml (data not shown). To avoid aggregation, prepolymer solutions for MN fabrication were prepared by mixing 1 volume of GelMA (30%) and 1 volume of PBAE/pDNA NP suspension (with 60 µg/ml pDNA), thus the total amount of DNA loaded into each MN/PBAE/DNA was 3 µg. After the prepolymer solution was cast into polydimethylsiloxane (PDMS) MN mold by centrifugation and drying, the fabricated patch 10 with MNs 14 were 600 µm in height, 300 µm in base diameter, and the distances between each MN were about 300 µm (FIGS. 4A, 4B and 4C). In addition, the incorporation of PBAE/DNA NPs 26 within the microneedles 14 of the patch 10 was visualized by SEM in which NPs 26 can be observed on the surface of the MN/PBAE/DNA (FIGS. 4D and 4E). During MN patch 10 fabrication, the stability of pDNA were maintained. The reagents and devices were sterilized and DNase free to keep the pDNA from degradation. The complexation with PBAE can further protect the pDNA. In addition, the nanoparticle underwent mild mechanic force during MN molding, which will not damage integrity of pDNA.

As a transdermal drug delivery MN device, the skin penetration properties and in vitro drug release profiles were further characterized. By changing the crosslinking time during the MN fabrication process, it was expected that the MN matrix crosslinking density can be leveraged to achieve tunable mechanical strength and drug release profiles. Therefore, different crosslinking periods (0 s, 5 s, 10 s, and 30 s) were tested. As shown in FIG. 4G, the MN/PBAE/DNA with longer crosslinking times required greater force to achieve the same amount of compression, indicating greater mechanical strength resulting from longer crosslinking. Moreover, the MN/PBAE/DNA showed similar mechanical properties compared to blank MNs (without drug) in samples crosslinked for 10 s, the optimal crosslinking period determined by in vitro transfection efficiency. Previous studies have determined that greater than 0.058 N per needle is required to force the MN into the skin. For the MN/PBAE/DNA crosslinked for 5 s or longer, the displacement under the required force (7.018 N for 11 × 11 MN array, 0.058 N per needle) was less than 0.2 mm, indicating that the MNs 14 are strong enough to penetrate the skin. To confirm the skin penetration capability of the MN/PBAE/DNA, the ex-vivo skin penetration was tested with mouse cadaver skin. After staining with trypan blue, a dye that binds to physically damaged cells, the hyperchromatic dot array on the skin indicated that MN/PBAE/DNA with 10 s crosslinking time can effectively penetrate the skin (FIG. 4F).

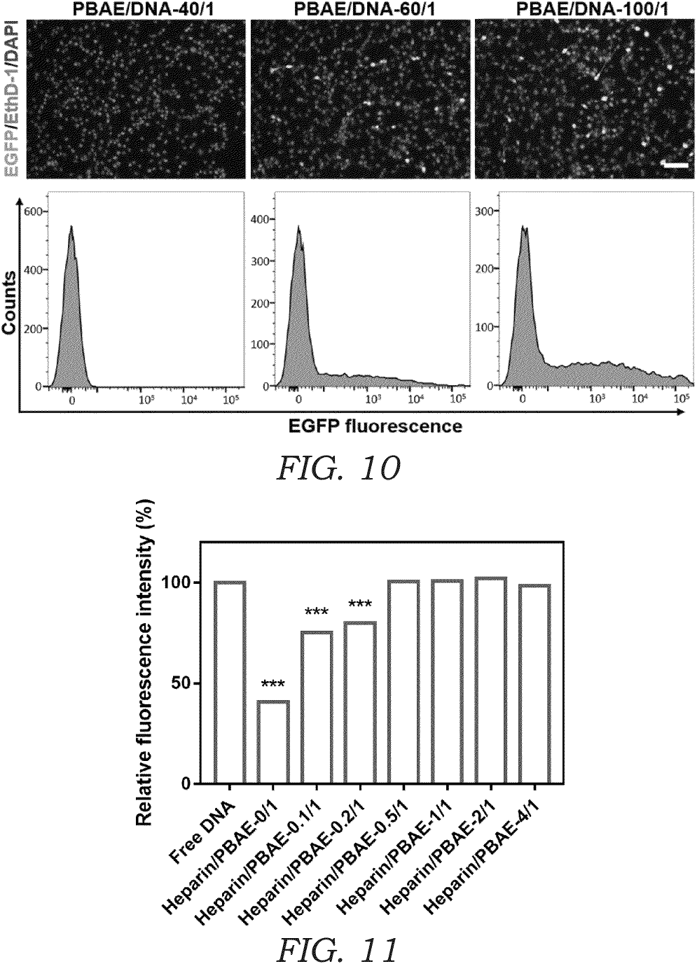

To characterize the release of the PBAE/DNA NPs 26 from the MN/PBAE/DNA, DNA staining agent (PicoGreen®) was used to quantify DNA released from the MN. However, the complexes formed by the binding of DNA with PBAE affected the binding and coloration of PicoGreen® (FIG. 11). Heparin is an anionic macromolecule, which can be used to destabilize the polyplexes, causing the DNA to dissociate from the PBAE. To explore the best dose of heparin used to free the DNA, heparin sulfate was used in a series of dilutions to dissociate DNA from PBAE/DNA NPs 26. Compared with the control group (free DNA without PBAE), a heparin:PBAE ratio over 0.5:1 was found to be able to disrupt the polyplexes and completely free the DNA (FIG. 11). To quantify DNA release from the MN/PBAE/DNA, applied heparin:PBAE was applied at a 1:1 mass ratio on samples with different crosslinking times. Similar to the mechanical tests, the release profiles of MN/PBAE/DNA with 4 different crosslinking times (0 s, 5 s, 10 s, and 30 s) were characterized. Furthermore, MN patches were soaked in PBS with or without type II collagenase to identify the release mechanism. In the presence of collagenase, MN/PBAE/DNA crosslinked for 5 s, 10 s, and 30 s released about 80%, 70%, and 50% of pDNA in 5 days, respectively (FIG. 4H). In contrast, the MN/PBAE/DNA only released less than 25% PBAE/DNA NPs 26 in 5 days in the absence of collagenase (FIG. 4I). This indicated that the release of PBAE/DNA NPs 26 from MN/PBAE/DNA progressed with the degradation of GelMA rather than the swelling of the GelMA hydrogel. Furthermore, longer crosslinking times prolonged the release of PBAE/DNA NPs 26 from the MNs 14, indicating the potential to tune the NP release profile by adjusting the crosslinking time.

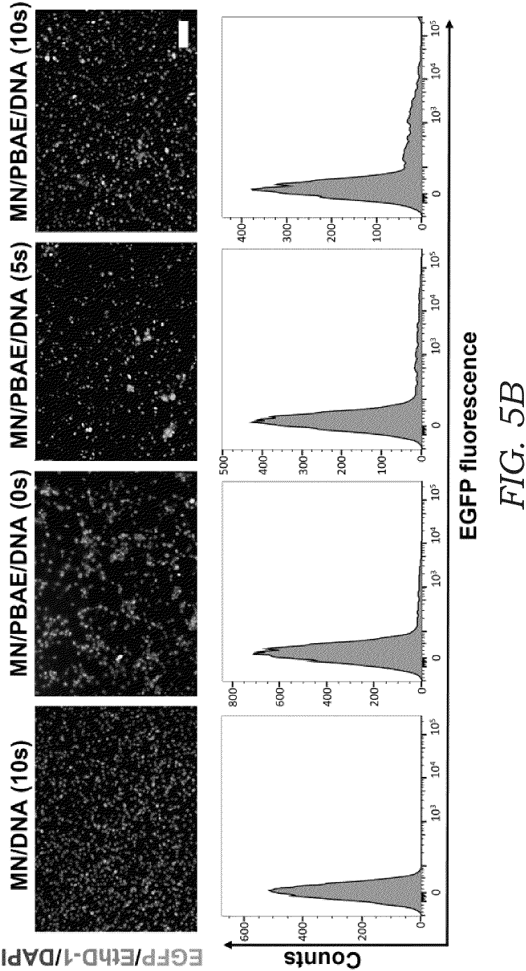

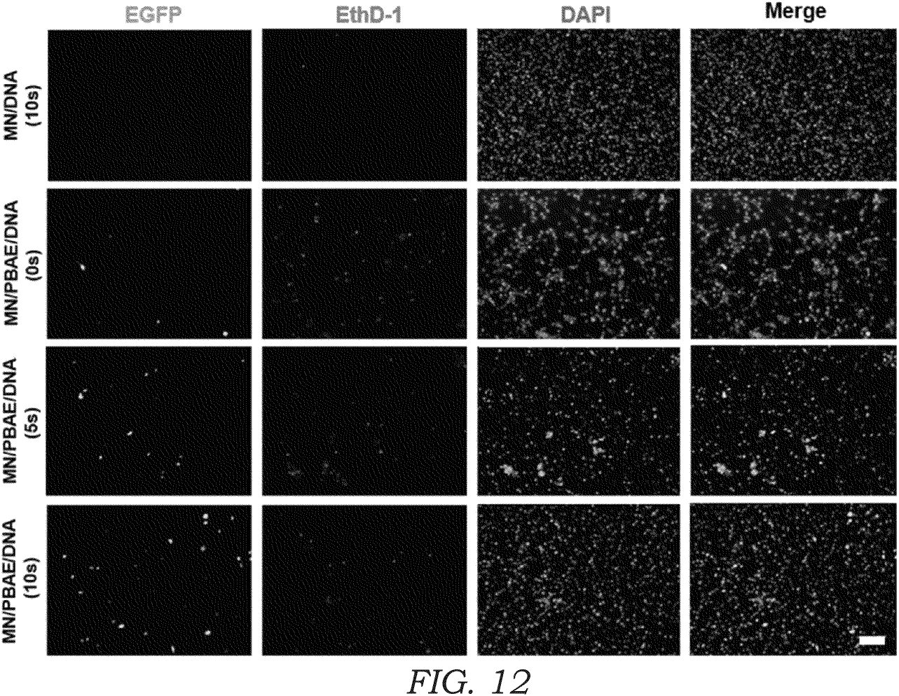

To validate the gene transfection efficiency of the MN/PBAE/DNA patch 10, MNs 14 with predetermined crosslinking times (0 s, 5 s, and 10 s) were incubated with NIH 3T3 cells in vitro. Blank MNs and MNs with free DNA (MN/DNA) were used as control groups. As shown in FIG. 5A, the MN patches 10 were placed in cell culture inserts (3 µm pore size), while cells were seeded at the bottom. Three days post-transfection, cells were stained with EthD-1 and DAPI to evaluate cytotoxicity and EGFP transfection rates by fluorescence imaging and flow cytometry. As shown in FIGS. 5B, 5 and FIG. 12, the MN/PBAE/DNA with a 10 s crosslinking time achieved efficient EGFP transfection (over 20%) in NIH 3T3 cells without causing severe cell death.

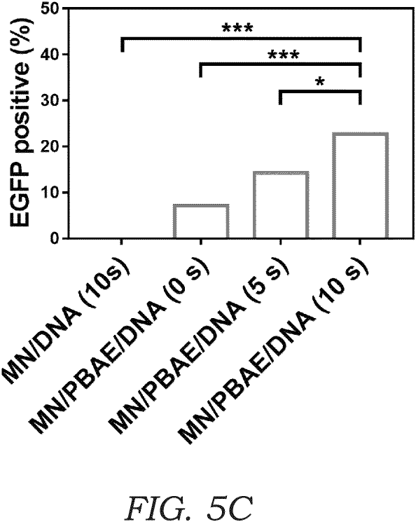

Following the 2D cell transfection experiment, 3D cell transfection was performed to better simulate in vivo transfection. A skin model was prepared by culturing NIH 3T3 cells in GelMA hydrogel. MN/PBAE/DNA with 10 s crosslinking time were inserted into the hydrogel to test the transfection efficacy (FIG. 6A). After insertion and incubation for 3 days, the cells were observed by confocal microscopy (FIG. 6B). Efficient EGFP expression can be observed in the MN/PBAE/DNA group but not in the MN/DNA group, which indicates that the MN-based gene delivery system can efficiently deliver plasmid DNA in a 3D environment.

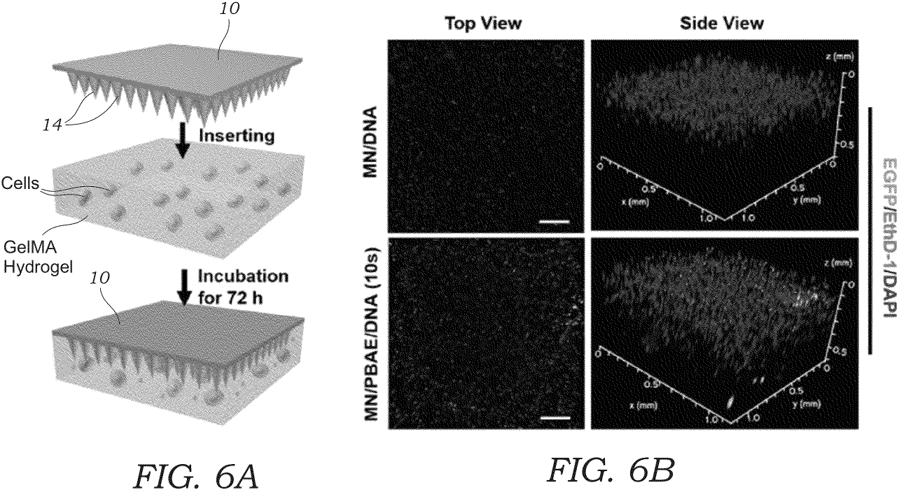

Next, a C57B1/6 mouse model was used to verify the in vivo gene delivery efficacy of the MN/PBAE/DNA. MN patches were applied to the dorsal skin of the mice on day 0 and the mice were sacrificed on day 3 for histological and immunofluorescence analysis. As shown in FIG. 7, all GelMA-based MN patches (Blank MN, MN/DNA, and MN/PBAE/DNA) effectively penetrated the epidermal layer of the skin and approached the dermal layer. In cross-sections of the skin harvest on day 3, a convexed epidermis with mild proliferation was observed in the area treated with the MNs 14. Upon observation with higher magnification, the local area of the MN application site did not show inflammation. In addition, there was no difference in the degree of inflammation with or without PBAE/pEGFP NP delivery. Immunofluorescence staining was further performed to confirm the EGFP delivery. As shown in the fluorescence images, green fluorescence signal was not observed in Blank MN or MN/DNA groups, while the green fluorescence signal was found within the dermal layer in mice treated with the MN/PBAE/DNA. The transfection rate of cells in the region of interest was estimated to be ~31%. In short, the MN/PBAE/DNA patch 10 enabled transdermal gene delivery without causing significant skin damage.

In summary, a MN patch 10 was developed for minimally invasive delivery of plasmid DNA to meet the increasing need for in situ local gene therapy. MNs 14 can penetrate the dermal layer of tissue 100 to enhance delivery efficiency. By using naturally derived GelMA as the MN matrix and non-viral synthetic polymer PBAE as gene carriers, high efficiency gene delivery was achieved both in vitro and in vivo. The GelMA matrix also serves as a protective scaffold for encapsulated PBAE/DNA NPs 26. The photocrosslinkability of GelMA can be leveraged to control both mechanical strength and DNA release profiles, which can be tuned for a range of applications. In addition, PBAE libraries with multiple polymer structures provides versatility in tailoring to different cell types to achieve targeted transfection, thus avoiding adverse effects in the surrounding tissue. By simply changing the delivered genes, it is expected that this platform can be leveraged for the delivery of transdermal gene therapies to address wound healing, skin cancer, and genetic skin diseases. In the future work, further evaluation of the in vivo distribution of the PBAE/DNA polyplex may be performed, testing the potential of the MN platform for gene delivery to other organs in a minimally invasive approach.

### Materials and Methods

### Materials

5-amino-1-pentanol, 1,4-butanediol diacrylate, dimethyl sulfoxide (DMSO), DMSO-d₆, agarose, gelatin from porcine skin, methacrylic anhydride, photoinitiator (Irgacure 2959), heparin sodium, and reagents for HE staining were purchased from Sigma-Aldrich (MO, USA). TAE buffer, loading dye, and DNA ladder were purchased from Bio-rad Laboratories (CA, USA). Cell culture media and reagents were purchased from Gibco Laboratories (NY, USA). NIH 3T3 cells were purchased from ATCC (VA, USA). Cell Counting Kit-8 (CCK-8) was purchased from Dojindo Molecular Technologies (Japan). Lipofectamine 3000, HEPES buffer, PicoGreen® dsDNA Assay Kit, collagenase (type II), DAPI, Live/Dead Kit and AlexaFluor 488 conjugated GFP antibody were purchased from ThermoFisher Scientific (NJ, USA). Antigen retrieval solution was purchased from Invitrogen (CA, USA).

### PBAE Synthesis

Poly(5-amino-1-pentanol-co-1,4-butanediol diacrylate) was synthesized according to protocols outlined in D.G. Anderson et al. and Y. Liu et al. (cited herein). Briefly, 5-amino-1-pentanol and 1,4-butanediol diacrylate (1.2:1 amine/diacrylate stoichiometric ratio) were weighed in a sample vial with a Teflon-lined screw cap and stirred at 1000 rpm at 90° C. for 24 hours, solvent-free. Afterwards, the polymers purified by precipitation with anhydrous diethyl ether. The suspension was vortexed for 20 s and centrifuged for 5 minutes at 1000 rpm at 4° C. The supernatant was removed following centrifugation, and the precipitate was washed again with ether. The polymer was maintained under vacuum with desiccant for 5 days to remove all remaining ether. The purified polymer was then dissolved in anhydrous DMSO to a concentration of 100 mg/mL and stored with desiccant at -20° C.

### ¹H-NMR

¹H-NMR spectra of the polymer were obtained using dimethyl suloxide-d₆ as the solvent. All measurements were performed from Brucker AV400 broad band FT NMR spectrometer with 64 scans at room temperature.

### DLS and TEM

The size and zeta potential of the PBAE/DNA NPs 26 were measured by DLS. Briefly, 2000 ng pEGFP was dissolved in 50 µL of 25 mM sodium acetate buffer (pH 5.0). Then, PBAE was dissolved in 50 µL of 25 mM sodium acetate buffer (pH 5.0) at different concentrations and added into the pEGFP solution to yield a mixture with different weight ratios of PBAE/DNA (1/1, 5/1, 10/1, 20/1, 40/1, 60/1, 80/1, 100/1). Complexes were formed after vortexing all mixtures for 15 s and subsequent incubation for 20 minutes. After that, 900 µL of 20 mM HEPES buffer was added into the PBAE/DNA mixtures. The size and zeta potential of the resultant PBAE/DNA NPs 26 were determined by DLS and the zeta potential analyzer (Zetasizer Nano-ZS, Malvern Instruments, Ltd., United Kingdom). The morphologies of the PBAE/DNA complexes were observed using a transmission electron microscope (TEM, Tecnai G2 20S-Twin, USA).

### Gel Electrophoresis

Gel electrophoresis was preformed to confirm the complexation between PBAE and DNA. PBAE/DNA complexes with 100 ng pEGFP and variable doses of PBAE with a predetermined ratio of PBAE/DNA were added into premade 8% agarose gel and run at 100 V for 40 min. Before loading, loading dye was added to each sample according to the manufacturer’s instructions. Following electrophoresis, the gel was visualized under UV using an imaging system (Bio-rad).

### Gene Transfection With PBAE/pDNA NPs In Vitro

Murine embryonic fibroblasts (NIH 3T3) were used to test pEGFP transfection efficacy with PBAE/DNA NPs 26. Cells were seeded in 96 well plates at a density of 5000 cells per well. After incubation for 24 h, the medium was changed to 100 µL Opti-mem with PBAE/DNA NPs 26 with 100 ng pEGFP and variables amounts of PBAE (PBAE/DNA: 0/1, 40/1, 60/1, 80/1 and 100/1). After a 4 h incubation, the medium was changed to 100 µL complete medium. Following an additional 24 h incubation, cells were stained with DAPI and EthD-1 and counted using fluorescence microscope and flow cytometry to assay viability. The commercialized non-viral gene vector, Lipofectamine 3000, was used according to the manufacturer’s protocol to generate the control group.

### Cytotoxicity Assay

To assay the cytotoxicity of PBAE/DNA NPs 26, the cell viability of transfected cells was measured using the CCK-8 assay. Briefly, cells were transfected with PBAE/pEGFP NPs 26 with different ratio of PBAE/DNA as previously described. Lipofectamine 3000 was used according to the manufacturer’s protocol as a control group. At pre-determined time points, the medium was removed, and complete medium supplemented with 10% CCK-8 reagent (v/v) was added. After a 2 h incubation, the absorbance of the medium was measured at 450 nm using a microplate reader (Varioskan Flash Multimode Reader, Thermo Scientific). Cell viability was reported as a relative percentage compared to untreated samples.

### Preparation of GelMA

GelMA was prepared according to a previously published protocol disclosed in J. W. Nichol et al., Biomaterials, 2010, 31, 5536-5544, which is incorporated herein by reference. Briefly, 10 g of type A gelatin from porcine skin was dissolved in 100 ml DPBS at 50° C. 0.25 ml methacrylic anhydride (MA) (0.25 volume%) was gradually stirred into the gelatin solution at a rate of 0.5 mL/min at 50° C. for 1 h. To stop the reaction, 500 ml of warm (40° C.) DPBS was added. Then, the unreacted salts and MA were removed by dialysis in 40° C. distilled water using 12-14 kDa cutoff dialysis tubing while stirring for one week. The resulting GelMA was lyophilized for one week and stored at -80° C. for further use.

### Preparation of MN/PBAE/DNA Patches

To fabricate the PBAE/pEGFP NP-loaded patches 10 with MNs 14, premade PBAE/pEGFP complexes with a PBAE/DNA ratio of 80/1 and photoinitiator (Irgacure 2959) were added into a GelMA solution to yield a final concentration of 15% GelMA with 30 µg/ml pEGFP and 0.5% photoinitiator. Then, 100 µL of solution was added into each MN mold. After centrifugation (3000 rpm for 5 min at 37° C.), the solution was exposed to 350 mW/cm² UV light for defined exposure durations (0, 5, 10, and 30 s). After dried in the dark, the MN-containing patch 10 was removed from the mold and kept at -20° C. until further use. In this procedure, the reagents and devices used were sterilized and DNase free to protect the pDNA from degradation.

### Sem

MNs 14 were coated with gold using a sputter coater (Pelco, SC-7) and the surface morphology was characterized using a field emission scanning electron microscope (ZEISS Supra 40VP SEM).

### Mechanical Properties of the MN/PBAE/DNA Patch

The mechanical strength of the MN/PBAE/DNA patch 10 with different UV exposure times was measured under dynamic force using a low-force mechanical testing system (5943 MicroTester, Instron, USA). During testing, the applied force and the corresponding deformation were recorded. In the test, MNs 14 were pressed against a stainless-steel plate at a speed of 0.5 mm/min with a maximum loading force of 50.0 N. To demonstrate that the MNs 14 could penetrate mouse skin, MNs 14 were pushed into the cadaver skin tissue 100 with 20 N of force for 30 seconds. Then, the penetrated skin was stained for 10 min using 0.5% trypan blue solution. After washing three times, trypan blue-stained samples were imaged which confirmed penetration.

### pDNA Release Properties

At the optimal ratio of heparin/PBAE, the amount of DNA in solution after dissociation should be approximate to the amount in solution before being complexed with PBAE. Heparin sulfate solutions with predetermined weight ratios between heparin and PBAE were added to the PBAE/DNA complexes and incubated for 15 min. Then, PicoGreen® was used (according to the manufacturer’s protocol) to quantify the amount of dissociated DNA. In order to detect the release of NPs 26 from the MNs 14, the MN/PBAE/DNA were soaked in DPBS with or without collagenase (2U/ml). At predetermined time points, a 50 µL suspension of each sample was incubated with heparin to dissociate the DNA, and PicoGreen® was added to quantify the concentration of DNA released from the MNs 14.

### Gene Transfection With the MN/PBAE/DNA in 2D Cell Models

NIH 3T3 cells were seeded in 12 wells plates at a density of 1×10⁵ cells per well. After incubation for 24 h, the medium was changed to medium with 2U/ml collagenase. Transwell inserts were placed in the well plate and sterilized MN/PBAE/DNA applied to the upper membrane of the Transwell. After 3 days of incubation, the cells were stained with DAPI and EthD-1 and counted using a fluorescence microscope and flow cytometry.

### Gene Transfection With MN/PBAE/DNA in 3D Cell Model

NIH 3T3 cells were 3D-cultured in a GelMA hydrogel matrix. Briefly, 200 µL of 10% GelMA solution with 2×10⁶ cells were added into a PDMS mold and crosslinked under UV light for 10 seconds. After one day of incubation, the previously prepared patch 10 of MN/PBAE/DNA (10 s crosslinking) was applied to the upper surface of the hydrogels. After further incubation for three days, the hydrogels (with cells) were imaged using a Leica Confocal SP8-STED/FLIM/FCS following staining with DAPI and EthD-1.

### In Vivo Transdermal Gene Transfection Model

All animal experiments were approved by the UCLA Animal Research Committee (UCLA ARC #2018-003-01E). Eighteen 7-week-old, C57BL/6J male mice (average weight: 20 grams) were purchased from Jackson Laboratory (Sacramento, CA, USA). All animals were treated in compliance with the National Research Council criteria as outlined in the “Guide for the Care of Laboratory Animals” prepared by the Institute of Laboratory Animal Resources and published by the National Institute of Health. At the day of application (Day 0), the patch 10 with MNs 14 were applied to the dorsum of the mice topically under inhalational anesthesia (1.5% isoflurane in 100% O₂). Blank MNs (n=6), MN/DNA (n=6) and MN/PBAE/DNA (3 µg pEGFP and 240 µg PBAE, n=6) were insert into the dorsum skin. On day 3, the mice were euthanized using CO₂ for further evaluation.

### Histological Analysis and Immunofluorescence Staining

Skin specimens including the patch 10 application site and surrounding skin were fixed in 10% neutral buffered formalin (Leica Biosystems, IL, USA). Then, skin samples were further processed for histological analysis and embedded in paraffin. Routine hematoxylin and eosin (HE) staining was conducted on 4 µm tissue sections. A Nikon inverted microscope was used to image the histology samples and AmScope image analysis software (AmScope, Irvine, CA, USA) was used for analysis. For immunostaining, the tissue sections were deparaffinized, antigen retrieved (heat-induced), permeabilized in PBST (0.3% Triton in PBS), and incubated with goat serum for 30 min. Then, the sections were incubated overnight at 4° C. with AlexaFluor 488 conjugated GFP antibody. The slides were rinsed with PBST and counterstained with DAPI for 5 min. The fluorescent images were imaged via Nikon Eclipse Ti-S Inverted Phase Contrast Fluorescent Microscope. The transfection rates of cells in interest region were analyzed using ImageJ (NIH).

### Statistics

All data are expressed as the mean ± standard deviation (SD). Statistical analyses were performed with Graph Pad Software (San Diego, CA, USA). The differences among the groups were analyzed by one-way ANOVA. Statistical significance was set at p<0.05.

While embodiments of the present invention have been shown and described, various modifications may be made without departing from the scope of the present invention. For example, any type of nucleic acid can be delivered using the patch. This includes RNA and DNA. In addition, while DNA encoding green fluorescent protein (GFP) was used for demonstration purposes, other genes may be delivered in a similar manner. The invention, therefore, should not be limited, except to the following claims, and their equivalents.

1. A patch for nucleic acid delivery into living tissue comprising:
a base or substrate having a plurality of microneedles extending away from the surface of the base, wherein the base and the plurality of microneedles are formed from crosslinked gelatin methacryloyl (GelMA) and the plurality of microneedles contain poly(β-amino ester) (PBAE)/nucleic acid nanoparticles therein.

2. The patch of claim 1, wherein upon application of the patch on living tissue, the plurality of microneedles release PBAE/nucleic acid nanoparticles into the living tissue.

3. The patch of claim 1, wherein nanoparticles comprise a ratio of PBAE to nucleic acid of between about 60/1 and about 80/1 on a weight basis.

4. The patch of claim 1, wherein the plurality of microneedles are biodegradable after insertion into the living tissue.

5. The patch of claim 1, wherein a rate of release of PBAE/nucleic nanoparticles from the plurality of microneedles is sustained over a period of hours after insertion of the patch into the living tissue.

6. The patch of claim 5, wherein PBAE/nucleic nanoparticles are released over a period of 100 hours or more.

7. The patch of claim 1, wherein the plurality of microneedles all have substantially the same lengths.

8-9. (canceled)

10. The patch of claim 1, wherein the nucleic acid comprises plasmid DNA (pDNA).

11. The patch of claim 1, wherein the nucleic acid comprises one or more of DNA, RNA, mRNA, siRNA, and miRNA.

12. The patch of claim 1, wherein the plurality of microneedles have a length of less than about 1.5 mm.

13. The patch of claim 1, wherein the plurality of microneedles have a length within the range of about 10 µm to about 1,500 µm.

14. The patch of claim 1, wherein the microneedles have diameter or width at a point of contact with the base that is less than about 500 µm.

15. The patch of claim 1, further comprising a backing material having an adhesive disposed thereon.

16. The patch of claim 1, wherein the plurality of microneedles have a shape or configuration of: a pyramid, cone, cylindrical, tapered tip, canonical, square base, or pentagonal-base canonical tip.

17. The patch of claim 1, wherein the PBAE/nucleic acid nanoparticles are well dispersed and not aggregated in the plurality of microneedles.

18. A patch for nucleic acid delivery into living tissue comprising:
a base or substrate having a plurality of microneedles extending away from the surface of the base, wherein the base and the plurality of microneedles are formed from crosslinked gelatin methacryloyl (GelMA) and the plurality of microneedles contain therein gene delivery nanoformulation(s) and nucleic acid.

19. The patch of claim 18, wherein the gene delivery nanoformulation(s) comprises nanoparticles, the nanoparticles forming a polyplex between a nucleic acid carrier and a nucleic acid.

20. The patch of claim 19, wherein the polyplex is formed by a nucleic acid and one of: poly(β-amino ester) (PBAE), poly(ethyleneimine), and poly(lysine).

21. The patch of claim 19, wherein the gene delivery nanoformulation(s) comprise one or more of: lipoplex, liposomes, peptides, and dendrimers.

22. A method of using the patch of claim 1 comprising placing the patch on live tissue of mammal such that the plurality of microneedles penetrates into the tissue.

23. The method of claim 22, wherein the tissue is selected from the group consisting of skin tissue, mucosal tissue, heart tissue, blood vessels, ocular tissue, gastrointestinal tissue, buccal tissue, muscle tissue, and vaginal tissue.

24. (canceled)
